(19) United States
(12) Patent Application Publication
(10) Pub. No.: US 2013/0041246 A1
(43) Pub. Date: Feb. 14, 2013

(54) LIGHT AND ULTRASOUND WHOLE BODY SCANNER

(71) Applicant: ROBERT L. SING, Santa Barbara, CA (US)

(72) Inventor: ROBERT L. SING, Santa Barbara, CA (US)

(21) Appl. No.: 13/412,405

(22) Filed: Mar. 5, 2012

Related U.S. Application Data
(60) Provisional application No. 61/521,175, filed on Aug. 8, 2011.

(51) Int. Cl. A61B 6/00

(52) U.S. Cl. 600/407

(57) ABSTRACT

The light and ultrasound whole body scanner produces an image of a patient's skin (visible light) and subcutaneous tissues (ultrasound). The scanner includes a turntable on which the patient stands during the scanning process. A computer operates the system and produces reports for later review. The report includes changes noted from previous scans. The report can be sent over the Internet, mailed or faxed to remote locations.

### CROSS-REFERENCE TO RELATED APPLICATION

This application claims the benefit of U.S. Provisional Patent Application Ser. No. 61/521,175, filed Aug. 8, 2011.

### BACKGROUND OF THE INVENTION

1. Field of the Invention

The present invention relates generally to medical imaging, and particularly to a light and ultrasound whole body scanner.

2. Description of the Related Art

The methods presently used for photographically or ultrasonically examining skin and subcutaneous organs of the human body, generally require a practitioner to hold a device over small portions of the patient while the patient may be in an uncomfortable physical position. This presents a problem for patient and practitioner.

Thus, a light and ultrasound whole body scanner solving the aforementioned problems is desired.

### SUMMARY OF THE INVENTION

The light and ultrasound whole body scanner produces an image of a patient's skin (visual light) and subcutaneous tissues (ultrasound). The scanner includes a turntable on which the patient stands during the scanning process. A computer operates the system and produces reports for later review. The report includes changes noted from previous scans. The report can be sent over the Internet, mailed or faxed to remote locations

These and other features of the present invention will become readily apparent upon further review of the following specification and drawings.

### BRIEF DESCRIPTION OF THE DRAWINGS

FIG. 1 is a diagrammatic front view of an exemplary light and ultrasound whole body scanner according to the present invention.

FIG. 2 is a flowchart showing steps of a method of using of the light and ultrasound whole body scanner of FIG. 1.

Similar reference characters denote corresponding features consistently throughout the attached drawings.

### DETAILED DESCRIPTION OF THE PREFERRED EMBODIMENTS

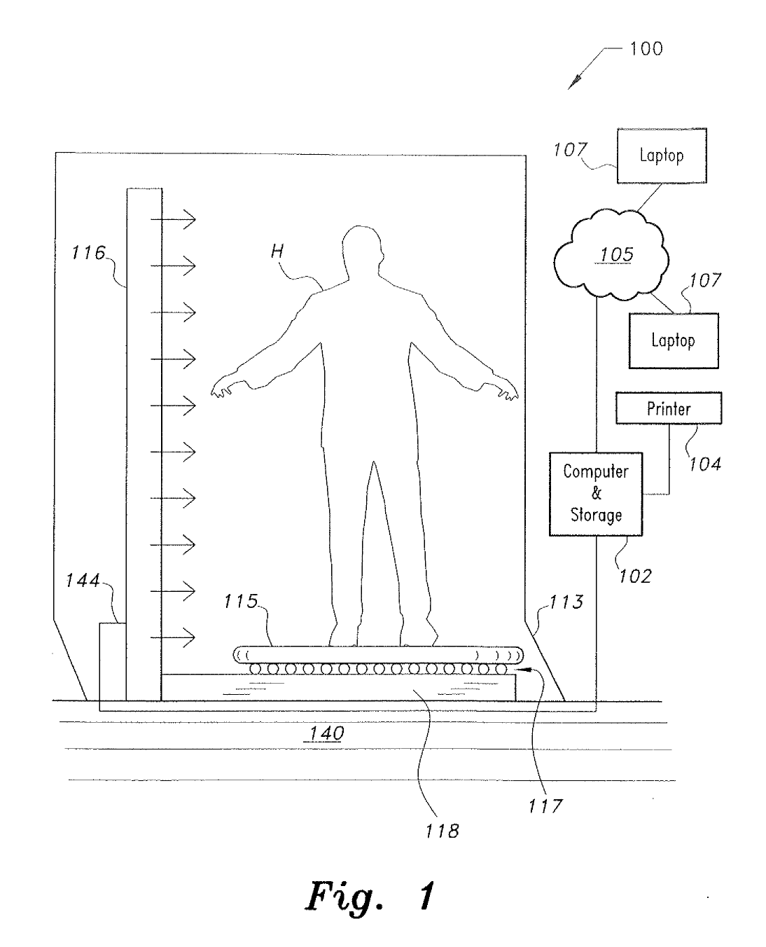

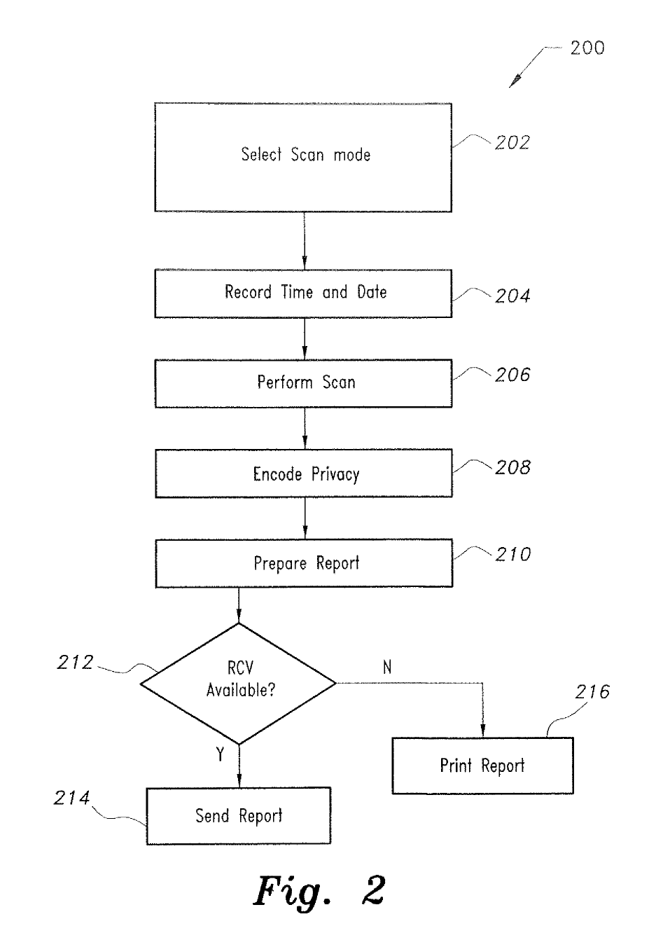

The light and ultrasound whole body scanner produces an image of a patient's skin (visual light) and subcutaneous tissues (ultrasound). As shown in FIGS. 1 and 2, the scanner system 100 includes a powered, kinetically rotatable turntable 115 that rotates over a ring-shaped bearing track 117. The base 118 of an elongate scanning member 116 and turntable/bearing assembly rests on the floor 140. The patient H stands on the slowly rotating turntable 115 during the scanning process. A curtain 113 surrounds the assembly for privacy of the patient H during the scanning process.

A computer 102 with data storage is connected to the scanning device 116 via electronic cabling 144 for exchange of control and data so that the computer 102 can operate the system 100 and produce reports, which may be printed on a printer 104 for later review. The data storage portion of the computer 102 is capable of storing and archiving historical scan data about a patient H and may employ a database to facilitate the historical scan data archiving feature.

Software run by computer 102 compares the historical data about the patient H with the data produced by the most recent scan, and the report can include changes noted from the historical (previous) scans. The report may include any interpretations the system 100 makes from the information available from scan histories, patient histories, and the like. The report can be sent over the Internet 105 to Web-enabled devices, such as exemplary remote laptop computer 107, or alternatively, the report can be printed out via printer 104 and mailed or faxed to remote locations.

As most clearly shown in FIG. 2, the scanning procedure 200 comprises a plurality of operating steps, beginning with scan mode selection step 202. Modes available for selection are visible light scan mode, and ultrasound scan mode. The scanner 116 (shown in FIG. 1) uses appropriate light for photographing the skin surface of patient H, noting pigmentation and raised or indented foci, or other variations of the skin surface. In the ultrasound scan mode, the system 100 can obtain information about subcutaneous tissues, including muscle, tendons, cartilage, and bone.

Step 204 records the date and time of the scan. Step 206 performs the scan under control of the computer 102. Step 208 encodes privacy in the data to conform to the privacy laws, e.g., HIPAA. The step 208 encoding step may include known techniques such as, e.g., data encryption. Step 210 prepares the report, while step 212 decides if a Web-enabled receiver has been designated and is available. If a Web-enabled receiver has been designated and is available, the report is sent over the Internet at step 214. Otherwise, the report is printed at step 216. Reports from the ultrasound mode of examination can be prepared with the skin photo mode, or as a separate report that does not include the skin examination mode,

It is to be understood that the present invention is not limited to the embodiments described above, but encompasses any and all embodiments within the scope of the following claims.

I claim:

1. A light and ultrasound whole body scanner, comprising:
a powered, kinetically rotatable turntable adapted for providing support for and rotating a patient standing thereon;
a combination ultrasound and visible light scanner for scanning the patient, the scanner being aimed at a target volume of space above the turntable dimensioned and configured for containing a whole body of the patient standing on the turntable;
an opaque curtain surrounding the scanner and the turntable, thereby providing privacy for the patient;
a computer with data storage electronically connected to the scanner, the computer controlling the scanner and recording scanning data about the patient on the data storage as the turntable is rotating with the scanner operating, the computer having an Internet connection for sending a report of the scanning data about the patient to a remote receiving station, a portion of the data storage being arranged into a database for archiving historical reports from historical scans about the patient, the computer including:
a first software program having means for comparing current data from a current scan to the historical reports from the historical scans about the patient; and
a second software program having means for sending a difference report highlighting differences between the data from the current scan and the data from the historical scans to the remote receiving station; and
means for selecting an operational mode of the scanner.

2. The light and ultrasound whole body scanner according to claim 1, further comprising means for tagging the scanning data associated with the patient with a storage record containing time and date of the scanning data.

3. The light and ultrasound whole body scanner according to claim 1, further comprising means for encoding privacy in the scanning data associated with the patient.

4. The light and ultrasound whole body scanner according to claim 3, wherein said report includes interpretations of the scan data and the patient's history, the interpretations being made by said first software program.

5. A light and ultrasound whole body scanner, comprising:
a powered, kinetically rotatable turntable adapted for providing support for and rotating a patient standing thereon;
a combination ultrasound and visible light scanner for scanning the patient, the scanner being aimed at a target volume of space above the turntable dimensioned and configured for containing a whole body of the patient standing on the turntable;
an opaque curtain surrounding the scanner and the turntable, thereby providing privacy for the patient;
a computer with data storage electronically connected to the scanner, the computer controlling the scanner and recording scanning data about the patient on the data storage as the turntable is rotating with the scanner operating, a portion of the data storage being arranged into a database for archiving historical reports from historical scans about the patient the computer including:
a first software program having means for comparing current data from a current scan to the historical reports from the historical scans about the patient; and
a second software program having means for sending a difference report highlighting differences between the data from the current scan and the data from the historical scans to the remote receiving station;
means for selecting an operational mode of the scanner; and
a printer connected to the computer.

6. The light and ultrasound whole body scanner according to claim 5, further comprising means for tagging the scanning data associated with the patient with a storage record containing time and date of the scanning data.

7. The light and ultrasound whole body scanner according to claim 5, further comprising means for encoding privacy in the scanning data associated with the patient.

8. The light and ultrasound whole body scanner according to claim 7, wherein said report includes interpretations of the scan data and the patient's history, the interpretations being made by said first software program.
